(19) United States
(12) Patent Application Publication
(10) Pub. No.: US 2013/0123729 A1
(43) Pub. Date: May 16, 2013

(54) ABSORBENT ARTICLE

(71) Applicants: Mari Minami, Kagawa (JP); Jun Kudo, Kagawa (JP); Hideyuki Kinoshita, Kagawa (JP); Yuji Takahashi, Kagawa (JP)

(72) Inventors: Mari Minami, Kagawa (JP); Jun Kudo, Kagawa (JP); Hideyuki Kinoshita, Kagawa (JP); Yuji Takahashi, Kagawa (JP)

(21) Appl. No.: 13/700,784

(22) PCT Filed: May 31, 2011
(86) PCT No.: PCT/JP2011/062471

§ 371 Date: Jan. 23, 2013

(30) Foreign Application Priority Data
May 31, 2010 (JP) 2010-125215

(51) Int. Cl. A61F 13/511; A61F 13/56

(52) U.S. Cl. CPC A61F 13/51104; A61F 13/5611
USPC 604/380

(57) ABSTRACT

An absorbent article having compressed grooves wherein the surface sheet is pressed to the absorbent body along the longitudinal direction are provided in the absorbent article. The compressed grooves are divided into at least a central compressed groove and posterior compressed grooves. In the longitudinal direction, the central compressed groove is provided to extend over a central line in the region of the absorbent article that contacts the excretion orifices. The posterior compressed grooves are provided posterior to the central line of the region contacting the excretion orifices in the longitudinal direction. The posterior ends of the central compressed groove are provided posterior to the anterior ends of the posterior compressed groove in the longitudinal direction. The posterior ends of the central compressed groove are provided to the outside of the anterior ends of the posterior compressed groove in the direction of width.

### TECHNICAL FIELD

The present invention relates to an absorbent article.

### BACKGROUND ART

Conventionally, there is known an absorbent article provided with a compressed portion configured to have compression from a topsheet to an absorber (for example, see Patent Literature 1).

Such a compressed portion increases a rigidity of the absorbent article, so that a twist or misalignment can be prevented from occurring even in a case where the absorbent article is worn for a long time.

### CITATION LIST

### Patent Literature

Patent Literature 1: Japanese Patent Application Publication No. 2007-7456

### SUMMARY OF INVENTION

However, the applicants faced the following problem as regard the above absorbent article.

In the absorbent article such as shown in FIG. 1 of Patent Literature 1, because the aforementioned compressed portion is provided without being divided in a longitudinal direction of the absorbent article, the absorbent article cannot get deformed flexibly according to irregularity of a wearer's body, thereby leading to a problem that the fitting into the wearer's body is diminished.

Further, in the absorbent article such as shown in FIG. 10 of Patent Literature 1, a gap part is provided in the longitudinal direction between divided pieces of the compressed portion, thereby leading to a problem that this gap part is possibly twisted to cause leakage of bodily fluid.

Thus, the present invention has been achieved in view of the aforementioned problem, and an object thereof is to provide an absorbent article which is capable of getting deformed flexibly according to irregularity of a wearer's body.

A first feature of the present invention is summarized as an absorbent article, comprising: a topsheet of liquid-permeable; a backsheet of liquid-impermeable; and an absorber disposed between the topsheet and the backsheet, wherein a compressed portion configured to have compression from the topsheet to the absorber is provided in a longitudinal direction of the absorbent article, the compressed portion is divided into at least a central compressed portion and a rear compressed portion, the central compressed portion is provided in the longitudinal direction so as to cross a central line of an excretion portion contact portion of the absorbent article, the rear compressed portion is provided posteriorly in the longitudinal direction from the central line of the excretion portion contact portion, a rear end of the central compressed portion is provided posteriorly in the longitudinal direction from a front end of the rear compressed portion, and the rear end of the central compressed portion is provided outwardly in a width direction of the absorbent article from the front end of the rear compressed portion.

### BRIEF DESCRIPTION OF DRAWINGS

FIG. 1 is a plan view of an absorbent article according to a first embodiment of the present invention, as seen from a skin contact surface side.

FIG. 2 shows a cross-sectional view taken along a line a-a and a cross-sectional view taken along a line b-b in the plan view of the absorbent article according to the first embodiment of the present invention, as seen from the skin contact surface side.

FIG. 3 is a plan view of the absorbent article according to the first embodiment of the present invention, as seen from a clothing contact surface side.

FIG. 4 is a view for explaining a method of manufacturing the absorbent article according to the first embodiment of the present invention.

FIG. 5 is a view for explaining a state in which the absorbent article according to the first embodiment of the present invention gets deformed when it is worn.

FIG. 6 is a plan view of an absorbent article according to a second embodiment of the present invention, as seen from a skin contact surface side.

### DESCRIPTION OF EMBODIMENTS

### First Embodiment of the Present Invention

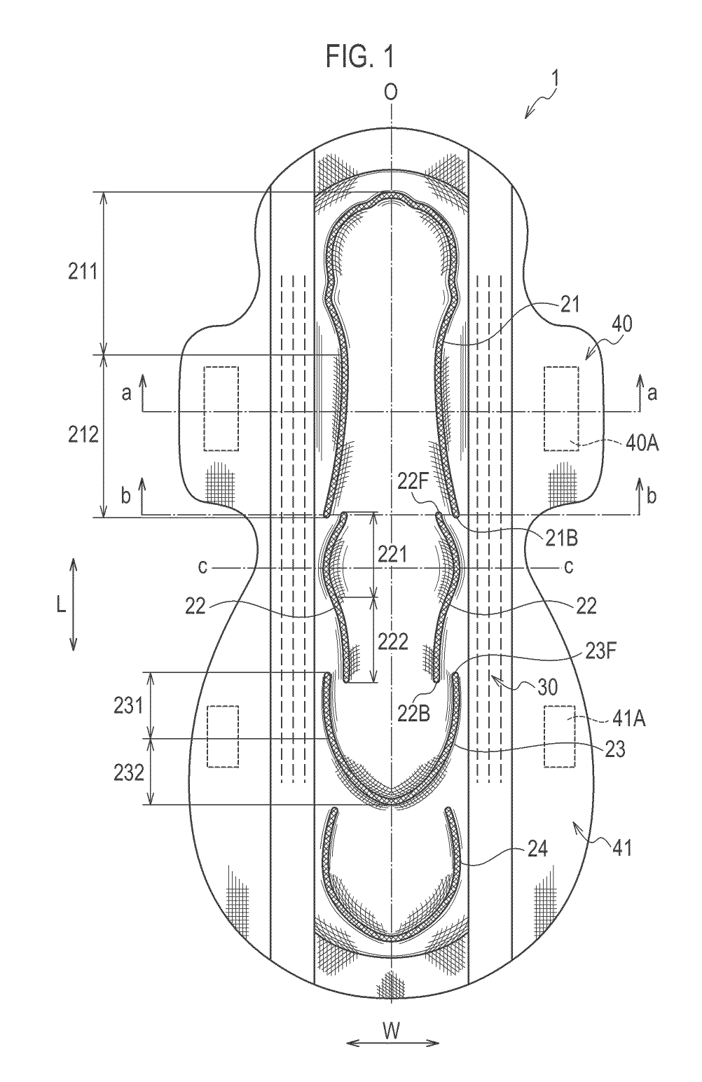

With reference to FIG. 1 to FIG. 5, an absorbent article 1 according to a first embodiment of the present invention will be described. For example, the absorbent article 1 according to the present embodiment is a paper diaper, an incontinence pad, sanitary napkin, or the like.

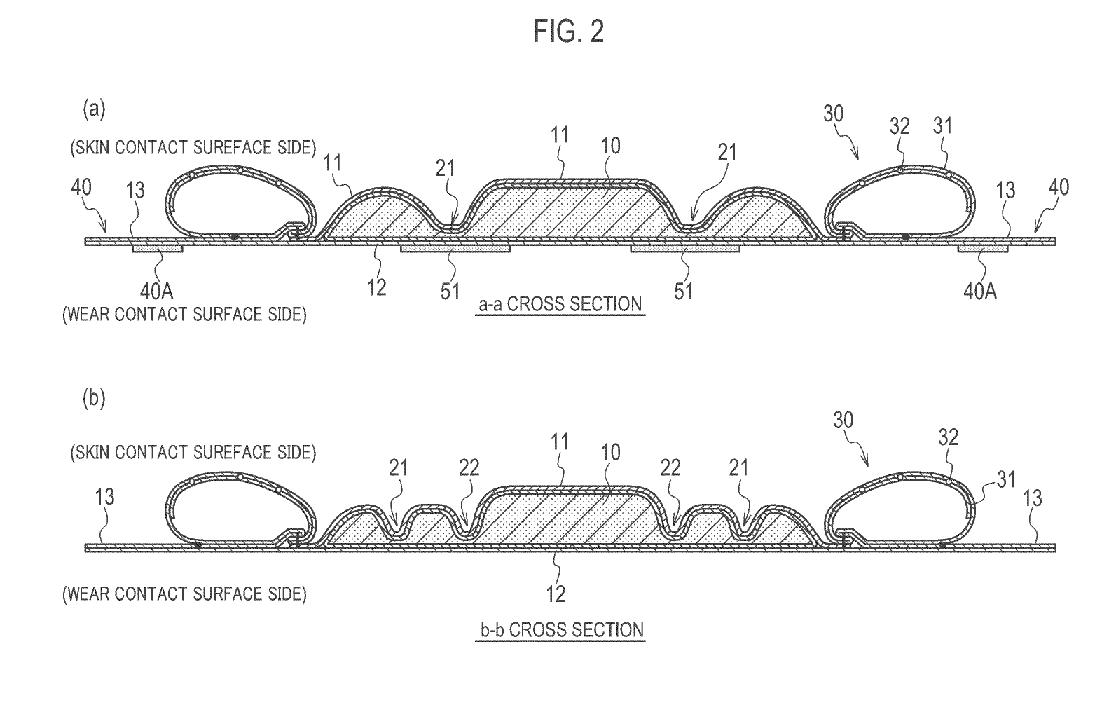

As shown in FIG. 1, FIG. 2(a), and FIG. 2(b), the absorbent article 1 according to the present embodiment is provided with a compressed portion configured to have compression from a topsheet 11 to an absorber 10 in a longitudinal direction L.

As shown in FIG. 1, this compressed portion is divided into a central compressed portion 21, a first rear compressed portion 22, a second rear compressed portion 23, and a third rear compressed portion 24.

The central compressed portion 21 is provided so as to cross a central line (a-a) of an excretion portion contact portion of the absorbent article 1, in the longitudinal direction L.

On the other hand, in the longitudinal direction L, the first rear compressed portion 22, the second rear compressed portion 23, and the third rear compressed portion 24 are provided posteriorly from the central line (a-a) of the excretion portion contact portion.

Note that the excretion portion contact portion of the absorbent article 1 according to the present embodiment is a portion which comes in contact with a vaginal opening of a wearer, in the absorbent article 1. For example, it is a portion which is disposed between two leg hole openings of underwear when the absorbent article 1 is worn on the underwear of the wearer.

Further, the absorbent article 1 according to the present embodiment may have a pair of central wing portions 40, which are formed so as to protrude outwardly in a width direction W at the excretion portion contact portion. Further, the absorbent article 1 according to the present embodiment may have a pair of rear wing portions 41 posteriorly from the central wing portions 40 in the longitudinal direction L.

Herein, when wearing the absorbent article 1 on the underwear, the wearer fixes the absorbent article 1 to the underwear by wrapping the central wing portions 40 around a crotch of the underwear and spreading the rear wing portions 41 on the underwear.

Therefore, for example, in a case where the central wing portions 40 are provided in the absorbent article 1 according to the present embodiment, the central line (a-a) of the aforementioned excretion portion contact portion corresponds to a central line of the central wing portions 40 in the longitudinal direction L.

Note that, in a case where the central wing portions 40 are not provided, when wearing the absorbent article 1 on the underwear, the wearer fixes the absorbent article 1 on the underwear by adjusting a position having the shortest length in the width direction W of the absorbent article 1, to the crotch of the underwear.

Therefore, for example, in a case where the central wing portions 40 are not provided in the absorbent article 1 according to the present embodiment, the central line (a-a) of the aforementioned excretion portion contact portion is a line which extends in the width direction W at a position having the shortest length in the width direction W of the absorbent article 1.

Further, in the longitudinal direction L, a rear end 21B of the central compressed portion 21 is provided posteriorly from a front end 22F of the first rear compressed portion 22.

Yet further, in the width direction W, the rear end 21B of the central compressed portion 21 is provided outwardly from the front end 22F of the first rear compressed portion 22.

Herein, the central compressed portion 21 may be formed so as to expand outwardly in the width direction W as it extends toward the rear end 21B at a rear portion 212 in the longitudinal direction L, or may be formed so as not to expand outwardly in the width direction W.

On the other hand, the central compressed portion 21 may be divided at a front portion 211 in the longitudinal direction L, or may be continuous without division.

Further, the first rear compressed portion 22 may be formed so as to narrow inwardly in the width direction W as it extends toward the front end 22F at a front portion 221 in the longitudinal direction L, or may be formed so as not to narrow inwardly in the width direction W.

Yet further, in the longitudinal direction L, a rear end 22B of the first rear compressed portion 22 may be provided posteriorly from a front end 23F of the second rear compressed portion 23.

In addition, in the width direction W, the rear end 22B of the first rear compressed portion 22 may be provided inwardly from the front end 23F of the second rear compressed portion 23.

Herein, the first rear compressed portion 22 may be formed so as to narrow inwardly in the width direction W as it extends toward the rear end 22B at a rear portion 222 in the longitudinal direction L, or may be formed so as not to narrow inwardly in the width direction W.

Note that the second rear compressed portion 23 may be formed so as to expand outwardly in the width direction W as it extends toward the front end 23F at a front portion 231 in the longitudinal direction L, or may be formed so as not to expand outwardly in the width direction W.

On the other hand, the second rear compressed portion 23 may be divided at a rear portion 232 in the longitudinal direction L, or may be continuous without division.

As shown in FIG. 2(a) and FIG. 2(b), the absorbent article 1 according to the present embodiment has a liquid-permeable topsheet 11, a liquid-impermeable backsheet 12, sidesheets 13 configuring the central wing portions 40, and an absorber 10 disposed between the topsheet 11 and the backsheet 13. Note that the absorbent article 1 according to the present embodiment may have a second sheet 12 between the topsheet 11 and the absorber 10.

The absorber 10 is configured by ground pulp, a superabsorbent polymer, and a core wrap for wrapping both. Herein, a macromolecular polymer is a granular polymer of a sodium acrylate copolymer.

Further, the core wrap is a sheet made from an arbitrary material which absorbs liquid. For example, as the core wrap, a tissue may be used, or an air-laid sheet formed by shaping a hydrophilic fiber or ground pulp into a sheet by an air-laid method may be used.

Yet further, the absorbent article 1 according to the present embodiment may be provided with a leakage-preventing wall 30 in the longitudinal direction L. This leakage-preventing wall 30 is configured by a leakage-preventing sheet 31 folded up in midair and an elastic member 32 disposed in a stretched state inside the leakage preventing sheet 31.

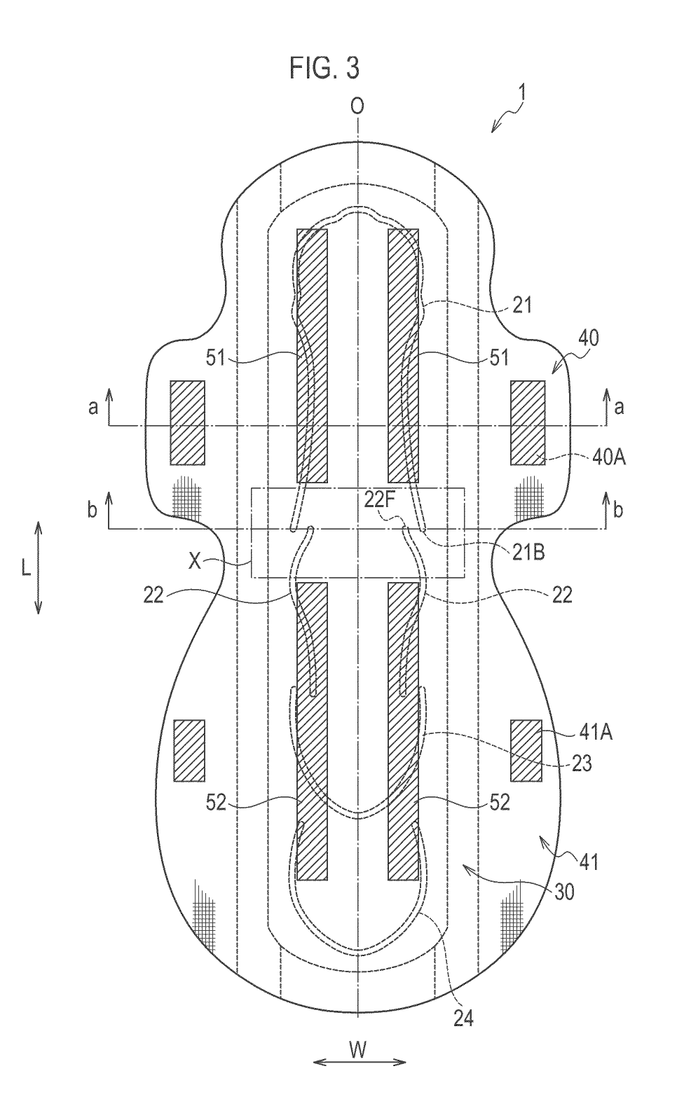

As shown in FIG. 3, in the absorbent article 1 according to the present embodiment, a clothing contact surface side of the backsheet is coated with an adhesive for adhering the clothing contact surface of the backsheet 12 to an inner surface of underwear.

For example, such an adhesive is coated on portions 40A inside the central wing portions 40 and portions 41A inside the rear wing portions 41.

Note that such an adhesive is not coated on an overlapping portion X but coated on a portion other than the overlapping portion X at the clothing contact surface side of the backsheet 12.

Herein, as shown in FIG. 3, at the clothing contact surface side of the backsheet 12, the overlapping portion X is a portion which extends in the width direction W from one side edge to the other side edge of the absorbent article 1, and which corresponds to a portion including the rear end 21 of the central compressed portion 21 and the front end 22F of the rear compressed portion 22.

With such a configuration, the absorbent article 1 and the underwear are not adhered to each other at the overlapping portion X, thereby making it easy to fit the absorbent article 1 into irregularity of the wearer's body without the influence of movement of the underwear in association with, e.g., turning over of the wearer.

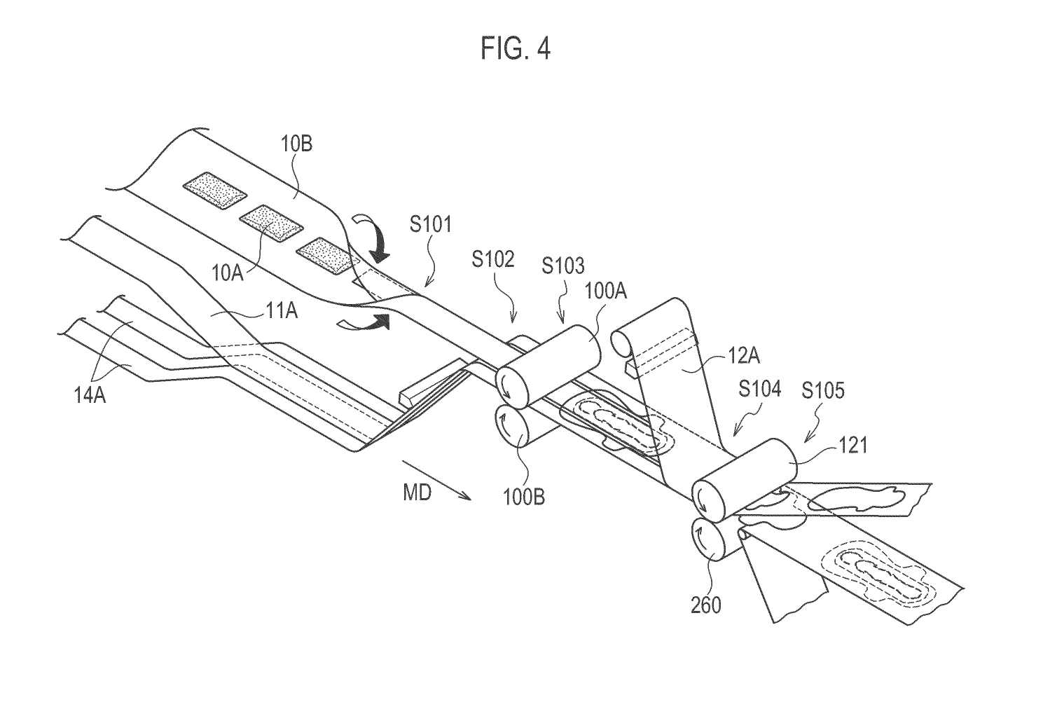

Next, some of the methods of manufacturing the absorbent article 1 according to the embodiment are described with reference to FIG. 4. Note that as far as the methods that are not described in FIG. 4 are concerned, the existing methods can be used.

As shown in FIG. 4, in step S101, the absorber 10 is generated by wrapping a core wrap 10B around a laminated body 10A of the ground pulp and the absorbent polymer.

In step S102, the absorber 20 is arranged on a web 11A for the topsheet 11 which is conveyed in continuation, and the web 11A for the topsheet 11 and the absorber 10 are bonded to each other using an adhesive.

In step S103, the web 11A for the topsheet 11 and the absorber 10, which are bonded together, are subjected to a compressing process using a press rollers 100A, 100B, thereby forming the central compressed portion 21, the first rear compressed portion 22, the second rear compressed portion 23, and the third rear compressed portion 24.

In step S104, a web 12A for the backsheet 12 is bonded to the web 11A for the topsheet 11 and the absorber 10 to which the compressing processing has been performed.

In step S105, the web 11A for the topsheet 11, the absorber 10, and the backsheet 12A for the backsheet 12, all of which have been bonded together, are cut in a predetermined size and shape using a product cutter, thereby generating the absorbent article 1.

According to the absorbent article 1 of the present embodiment, because the central compressed portion 21 and the rear compressed portions 22 to 24 are divided so that force transmitted from the crotch portion of the wearer to the central compressed portion 21 is not transmitted directly to the rear compressed portions 22 to 24, the absorbent article 1 is capable of getting deformed flexibly according to the irregularity of the wearer's body.

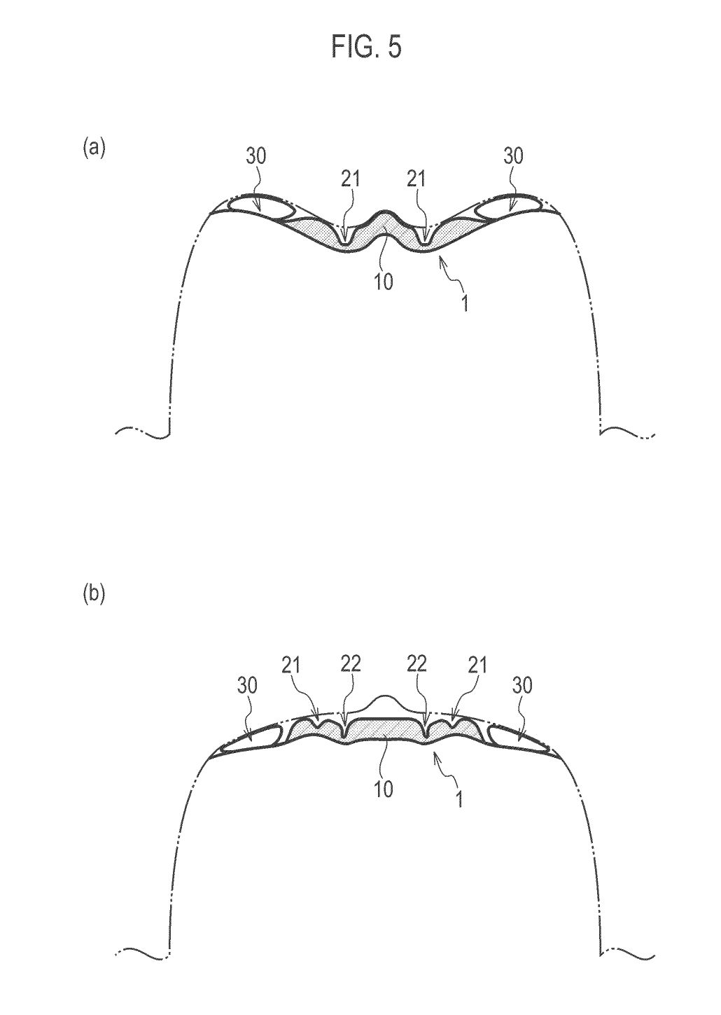

Specifically, at a neighboring portion of the central line (a-a) of the excretion portion contact portion, that is, at a portion which comes in contact with the crotch portion of the wearer, as shown in FIG. 5(a), the side edge of the absorber 10 is configured to rise along the central compressed portion 21 by force from the crotch portion of the wearer, so that the absorbent article 1 can be fit into the irregularity of the wearer's body without any gaps.

On the other hand, at the rear side in the longitudinal direction L of the central line (a-a) of the excretion portion contact portion (for example, a line (c-c) in the width direction shown in FIG. 1), that is, at a portion which comes in contact with the buttocks of the wearer, as shown in FIG. 5(b), the force from the crotch portion of the wearer is transmitted indirectly to the rear compressed portions 22 to 24 through the central compressed portion 21, so that the side edge of the absorber 10 does not rise and the absorbent article 1 can be fit into the irregularity of the wearer's body without any gaps.

According to the absorbent article 1 of the present embodiment, in the longitudinal direction L, the rear end 21B of the central compressed portion 21 is provided posteriorly from the front end 22F of the first rear compressed portion 22, so that the bodily fluid flowing in the width direction W can be absorbed by both of the central compressed portion 21 and the first rear compressed portion 22, thereby being able to prevent leakage of the bodily fluid in the width direction W.

According to the absorbent article 1 of the present embodiment, in the longitudinal direction L, the rear end 21B of the central compressed portion 21 is provided posteriorly from the front end 22F of the first rear compressed portion 22, so that in the longitudinal direction L, a gap part does not exist between the central compressed portion 21 and the first rear compressed portion 22, thereby being able to prevent leakage of the bodily fluid due to a twist of such a gap part.

According to the absorbent article 1 of the present embodiment, in a case where the central compressed portion 21 is formed so as to expand outwardly in the width direction W as it extends toward the rear end 21B at the rear portion 212 in the longitudinal direction L, at the central compressed portion 21, the force from the crotch portion of the wearer is guided outwardly in the width direction W, thereby further ensuring that the force is not transmitted to the rear compressed portions 22 to 24.

According to the absorbent article 1 of the present embodiment, in a case where the rear compressed portion 22 is formed so as to narrow inwardly in the width direction W as it extends toward the front end 22F at the front portion 221 in the longitudinal direction L, it is more difficult to transmit the force from the crotch portion of the wearer through the central compressed portion 21 to the rear compressed portion 22.

According to the absorbent article 1 of the present embodiment, the first rear compressed portion 22 narrows inwardly in the width direction W as it extends toward the rear end 22B at the rear portion 222 in the longitudinal direction L, so that the absorbent article 1 gets easily deformed in the longitudinal direction L of the absorbent article 1 while being inhibited from getting deformed in the width direction W, thereby making it easy to fit the absorbent article 1 into a curve of the wearer's buttocks.

### Second Embodiment of the Present Invention

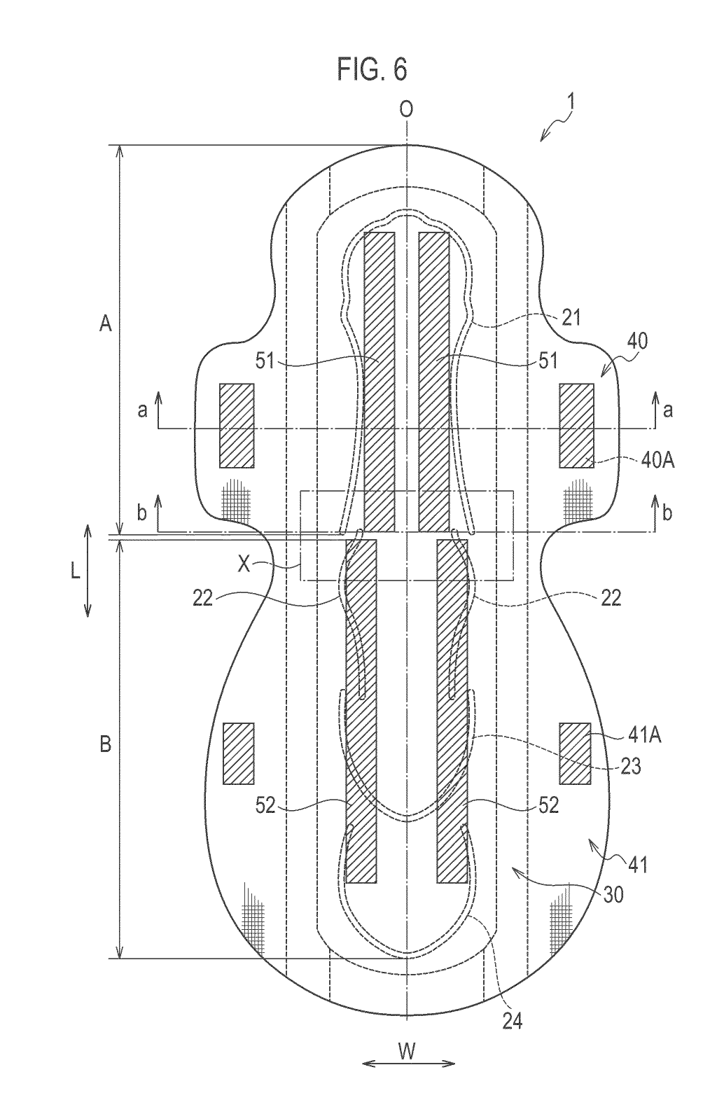

With reference to FIG. 6, the absorbent article 1 according to a second embodiment of the present invention will be described. The absorbent article 1 according to the present embodiment will be described while focusing on the differences from the absorbent article 1 according to the aforementioned first embodiment.

As shown in FIG. 6, in the absorbent article 1 according to the present invention, at a central portion A, an adhesive for adhering the clothing contact surface of the backsheet 12 to the inner surface of the underwear is not coated on a portion corresponding to an outward in the width direction W of the central compressed portion 21 but coated on a portion corresponding to an inward in the width direction W of the central compressed portion 21.

Herein, at the central portion A, as long as such an adhesive is coated on at least part of the portion corresponding to the inward in the width direction W of the central compressed portion 21, the adhesive may be coated on, for example, a portion in an substantially rectangular shape within the portion corresponding to the inward in the width direction W of the central compressed portion 21, as shown in FIG. 6.

Note that at the central portion A, the adhesive may be coated on at least part of a portion corresponding to the central compressed portion 21.

On the other hand, as shown in FIG. 6, in the absorbent article 1 according to the present embodiment, at a rear portion B, the adhesive is not coated on a neighboring portion of a central line O in the width direction but coated on an outward in the width direction W of the neighboring portion of the central line O in the width direction W.

Herein, at the rear portion B, as long as such an adhesive is coated outwardly in the width direction W from the neighboring portion of the central line O in the width direction W, the adhesive portion may be coated on any of a portion corresponding to the inward in the width direction W of the first rear compressed portion 22, a portion corresponding to the outward in the width direction W of the first rear compressed portion 22, and a portion corresponding to the first rear compressed portion 22.

For example, as shown in FIG. 6, at the rear portion B, the adhesive may be coated across the portion corresponding to the inward in the width direction W of the first rear compressed portion 22, the portion corresponding to the outward in the width direction W of the first rear compressed portion 22, and the portion corresponding to the first rear compressed portion 22.

Further, as shown in FIG. 6, a coating position of the adhesive at the rear portion B may be provided outwardly in the width direction W from a coating position of the adhesive at the central portion A.

Herein, the central portion A is a portion corresponding to a portion provided with the central compressed portion 21 at the clothing contact surface side of the backsheet 12, and the rear portion B is a portion provided outwardly in the longitudinal direction L from the central portion A.

According to the absorbent article 1 of the present embodiment, it becomes easy for the side edge of the absorber 10 to rise at the central portion A and for the neighboring portion of the central line O in the width direction W to get deformed at the rear portion B, so that the absorbent article 1 can be fit into the irregularity of the wearer's body without any gaps.

### Modification

The absorbent article 1 according to the first and second embodiments described above may be configured so that the leakage-preventing wall 30 is provided within a portion (for example, the portion corresponding to the central portion A in FIG. 6) provided with the central compressed portion 21 in the longitudinal direction L.

With such a configuration, contraction of the leakage-preventing wall 30 can be completed within the portion provided with the central compressed portion 21, thereby being able to reduce the linkage between the central compressed portion 21 and the first rear portion.

Thus, the present invention has been explained in detail by using the above-described embodiments; however, it is obvious that for persons skilled in the art, the present invention is not limited to the embodiments explained herein. The present invention can be implemented as corrected and modified modes without departing from the gist and the scope of the present invention defined by the claims. Therefore, the description of the specification is intended for explaining the example only and does not impose any limited meaning to the present invention.

In addition, the entire content of Japanese Patent Application No. 2010-125215 (filed on May 31, 2010) is incorporated in the present specification by reference.

### INDUSTRIAL APPLICABILITY

As described above, it is possible to provide the absorbent article which is capable of getting deformed flexibly according to irregularity of a wearer's body.

### REFERENCE SIGNS LIST

1 . . . Absorbent article
10 . . . Absorber
11 . . . Topsheet
12 . . . Backsheet
13 . . . Sidesheet
21 . . . Central compressed portion
22 . . . First rear compressed portion
23 . . . Second rear compressed portion
24 . . . Third rear compressed portion
30 . . . Leakage-preventing wall
31 . . . Leakage-preventing sheet
40 . . . Central wing portion
41 . . . Rear wing portion

1. An absorbent article, comprising:
a topsheet of liquid-permeable;
a backsheet of liquid-impermeable; and
an absorber disposed between the topsheet and the backsheet, wherein
a compressed portion configured to have compression from the topsheet to the absorber is provided in a longitudinal direction of the absorbent article,
the compressed portion is divided into at least a central compressed portion and a rear compressed portion,
the central compressed portion is provided in the longitudinal direction so as to cross a central line of an excretion portion contact portion of the absorbent article,
the rear compressed portion is provided posteriorly in the longitudinal direction from the central line of the excretion portion contact portion,
a rear end of the central compressed portion is provided posteriorly in the longitudinal direction from a front end of the rear compressed portion, and
the rear end of the central compressed portion is provided outwardly in a width direction of the absorbent article from the front end of the rear compressed portion.

2. The absorbent article according to claim 1, wherein the central compressed portion is formed so as to expand outwardly in the width direction as it extends toward the rear end at a rear portion in the longitudinal direction.

3. The absorbent article according to claim 1, wherein the rear compressed portion is formed so as to narrow inwardly in the width direction as it extends toward the front end at a front portion in the longitudinal direction.

4. The absorbent article according to claim 1, wherein
the rear compressed portion is divided into at least a first rear compressed portion and a second rear compressed portion, and
a rear end of the first rear compressed portion is provided posteriorly in the longitudinal direction and inwardly in the width direction with respect to a front end of the second rear compressed portion.

5. The absorbent article according to claim 1, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

6. The absorbent article according to claim 1, wherein
a clothing contact surface side of the backsheet is provided with a central portion corresponding to a portion provided with the central compressed portion, and a rear portion provided posteriorly in the longitudinal direction from the central portion,
at the central portion, an adhesive for adhering a clothing contact surface of the backsheet to an inner surface of underwear is not coated on a portion corresponding to an outward in the width direction of the central compressed portion but coated on a portion corresponding to an inward in the width direction of the central compressed portion, and
at the rear portion, the adhesive is not coated on a neighboring portion of a central line in the width direction but coated on an outward in the width direction of the neighboring portion of the central line in the width direction.

7. The absorbent article according to claim 1, wherein
central wing portions configured to protrude outwardly at both sides in the width direction are provided, and
the central line of the excretion contact portion corresponds to a central line of the central wing portions in the longitudinal direction.

8. The absorbent article according to claim 1, wherein
central wing portions configured to protrude outwardly at both sides in the width direction are not provided, and
the central line of the excretion contact portion is a line which extends in the width direction at a position having the shortest length in the width direction of the absorbent article.

9. The absorbent article according to claim 2, wherein the rear compressed portion is formed so as to narrow inwardly in the width direction as it extends toward the front end at a front portion in the longitudinal direction.

10. The absorbent article according to claim 2, wherein
the rear compressed portion is divided into at least a first rear compressed portion and a second rear compressed portion, and
a rear end of the first rear compressed portion is provided posteriorly in the longitudinal direction and inwardly in the width direction with respect to a front end of the second rear compressed portion.

11. The absorbent article according to claim 3, wherein
the rear compressed portion is divided into at least a first rear compressed portion and a second rear compressed portion, and
a rear end of the first rear compressed portion is provided posteriorly in the longitudinal direction and inwardly in the width direction with respect to a front end of the second rear compressed portion.

12. The absorbent article according to claim 9, wherein
the rear compressed portion is divided into at least a first rear compressed portion and a second rear compressed portion, and
a rear end of the first rear compressed portion is provided posteriorly in the longitudinal direction and inwardly in the width direction with respect to a front end of the second rear compressed portion.

13. The absorbent article according to claim 2, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

14. The absorbent article according to claim 3, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

15. The absorbent article according to claim 4, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

16. The absorbent article according to claim 9, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

17. The absorbent article according to claim 10, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

18. The absorbent article according to claim 11, wherein
a clothing contact surface side of the backsheet is provided with an overlapping portion as a portion extending in the width direction from one side edge to the other side edge of the absorbent article as well as a portion corresponding to a portion including the rear end of the central compressed portion and the front end of the rear compressed portion, and
an adhesive for adhering a clothing contact surface of the backsheet to an inner surface of underwear is not coated on the overlapping portion but coated on a portion other than the overlapping portion at the clothing contact surface side of the backsheet.

19. The absorbent article according to claim 2, wherein
a clothing contact surface side of the backsheet is provided with a central portion corresponding to a portion provided with the central compressed portion, and a rear portion provided posteriorly in the longitudinal direction from the central portion,
at the central portion, an adhesive for adhering a clothing contact surface of the backsheet to an inner surface of underwear is not coated on a portion corresponding to an outward in the width direction of the central compressed portion but coated on a portion corresponding to an inward in the width direction of the central compressed portion, and
at the rear portion, the adhesive is not coated on a neighboring portion of a central line in the width direction but coated on an outward in the width direction of the neighboring portion of the central line in the width direction.

20. The absorbent article according to claim 3, wherein
a clothing contact surface side of the backsheet is provided with a central portion corresponding to a portion provided with the central compressed portion, and a rear portion provided posteriorly in the longitudinal direction from the central portion,
at the central portion, an adhesive for adhering a clothing contact surface of the backsheet to an inner surface of underwear is not coated on a portion corresponding to an outward in the width direction of the central compressed portion but coated on a portion corresponding to an inward in the width direction of the central compressed portion, and
at the rear portion, the adhesive is not coated on a neighboring portion of a central line in the width direction but coated on an outward in the width direction of the neighboring portion of the central line in the width direction.
